(19) United States
(12) Patent Application Publication
(10) Pub. No.: US 2010/0069749 A1
(43) Pub. Date: Mar. 18, 2010

(54) IN VIVO DOSIMETRY DEVICE

(75) Inventors: Guo-Neng Lu, Villeurbanne (FR); Patrick Pittet, Fontaines Saint Martin (FR); Jean-Marc Galvan, Villeurbanne (FR)

Correspondence Address: DENNISON, SCHULTZ & MACDONALD, 1727 KING STREET, SUITE 105, ALEXANDRIA, VA, 22314, US

(73) Assignees: UNIVERSITE CLAUDE BERNARD LYON I, Villeurbanne Cedex (FR); CENTRE HOSPITALIER UNIVERSITAIRE DE GRENOBLE, Grenoble Cedex 9 (FR); UNIVERSITE JOSEPH FOURIER GRENOBLE I, Gieres (FR)

(21) Appl. No.: 12/523,612

(22) PCT Filed: Jan. 30, 2008
(86) PCT No.: PCT/FR2008/050153

§ 371 Date: Oct. 28, 2009

(30) Foreign Application Priority Data
Jan. 30, 2007 (FR) 0752962

(51) Int. Cl. A61B 6/00

(52) U.S. Cl. 600/436

(57) ABSTRACT

A device for in vivo dosimetry, the device comprising:
a miniature probe (1) comprising at least:
   a radioluminescent material (3) that emits a radioluminescence signal of intensity that is a function of the high-energy radiation irradiating said material; and
   an optical fiber (4, 16) receiving the luminescence signal and conveying it to a luminescence detector system (14); and
a luminescence detector system (14);
the device being characterized in that the radioluminescent material (3) is gallium nitride (GaN) that emits a luminescence signal at least in a narrow band BE, and in that the luminescence detector system (14) includes an optical device (18) enabling the narrow emission band of gallium nitride to be selected.

The present invention relates to the technical field of invasive or non-invasive in vivo dosimetry for external radiotherapy or radiodiagnosis.

The invention relates more precisely to a dosimetry device including a miniature probe serving to measure a dose of high-energy radiation.

In the preferred field of in vivo dosimetry in radiotherapy, there is a need to measure the high-energy radiation that appears in anatomical zones that are irradiated voluntarily, and/or in peripheral anatomical zones that are sensitive to radiation. This in vivo measurement serves to verify the delivered dose and/or to assess the impact of high-energy radiation in the surroundings of the irradiated zone.

In the state of the art, a first category of dosimetry is known that is of the semiconductor type, and it is described for example in patents U.S. Pat. No. 5,959,075, U.S. Pat. No. 5,587,199, or in the article summarizing the state of the art and entitled “Electronic dosimetry in radiation therapy” (Radiation Measurements, Volume 41, Supplement 1, Dec. 1, 2006, pp. S134-S153). That type of dosimetry comprises a detection cell containing a semiconductor device such as a diode or a metal oxide semiconductor field effect transistor (MOSFET) enabling high-energy photons or particles to be converted into electrons. The use of a radioelectric type of converter cell requires electrical interconnections to be implemented that are highly penalizing in terms of miniaturization and immunity to electromagnetic disturbances. Furthermore, that type of dosimetry presents the drawback of being sensitive to the orientation of the detection cell relative to the high-energy beam.

A second category of dosimeters based on insulating scintillating materials is also known, in particular from patent FR 2 822 239 and from the article summarizing the state of the art entitled “Optically stimulated luminescence and its use in medical dosimetry” (Radiation Measurements, Volume 41, Supplement 1, Dec. 1, 2006, pp. S78-S99). As a detection cell, that type of dosimeter includes an insulating scintillating material that converts high-energy photons or particles into photons that may be ultraviolet, visible, or infrared. The luminescence signal emitted by that insulating scintillating material is conveyed by an optical fiber to a photodetector that performs photoelectric conversion. It should be observed that the conversion of high-energy photons or particles into ultraviolet, visible, or infrared photons makes use of radioactive recombination centers in the forbidden band, which leads to conversion efficiency that is relatively low. To increase this conversion efficiency, it is known to have recourse to thermal or optical stimulation. Nevertheless, implementing thermal or optical stimulation makes the provision of the dosimeter more complex and impedes miniaturization of such a dosimeter.

The state of the art also includes dosimetry probes based on scintillating fibers that enable detection to be achieved without having recourse to optical or thermal stimulation, as can be seen in particular from the article entitled “Plastic scintillation dosimetry and its application to radiotherapy” (Radiation Measurements, Volume 41, Supplement 1, Dec. 1, 2006, pp. S124-S133). Nevertheless, the low efficiency of the luminescence enables only limited miniaturization to be achieved for the scintillating elements.

In another technical field, and in particular from patent U.S. Pat. No. 6,643,538, the use of II-VI semiconductor materials CdTe, CdZnTe, HgI₂ is described for their scintillation properties for guiding a surgical endoscopic probe to a radio-marked tube. That probe includes an optical channel that increases the diameter of the probe, and thus prevents a miniaturized probe being obtained. Furthermore, the semiconductor materials recommended have an atomic number that is high, which will disturb the uniformity of the irradiation dose in the vicinity of the scintillation material, thereby limiting the advantage of such materials for in situ in vivo dosimetry.

The object of the invention is thus to remedy the drawbacks of the prior art by proposing a novel device for in vivo dosimetry that presents low cost, while including a miniature probe suitable for performing measurements that are accurate.

To achieve this object, the invention provides a device for in vivo dosimetry, the device comprising:
a miniature probe comprising at least:
   a radioluminescent material that emits a radioluminescence signal of intensity that is a function of the high-energy radiation irradiating said material; and
   an optical fiber receiving the luminescence signal and conveying it to a luminescence detector system; and
a luminescence detector system.

According to the invention, the radioluminescent material is gallium nitride that emits a luminescence signal at least in a narrow band, and the luminescence detector system includes an optical device enabling the narrow emission band of gallium nitride to be selected.

In an advantageous embodiment, the GaN radioluminescent material is doped specifically so that it emits essentially in the narrow emission band (BE).

In an embodiment, the GaN radioluminescent material is placed in a detection cavity mounted on the end of an optical fiber or made at one of the ends of an optical fiber in order to form an invasive probe.

In another embodiment, the optical fiber includes a tubular covering for protecting optical cladding that contains the core of the optical fiber, the core of the fiber and optionally the cladding being removed over an end portion of the optical fiber in order to constitute the cavity for receiving the radioluminescent material, this cavity being closed by a protective material.

In order to perform differential measurement, the probe includes a reference optical fiber identical to the fiber connected to the luminescent material, but not connected to any radioluminescent material, the optical fiber and the reference optical fiber being connected via the optical selector device to two identical photomultiplier tubes to enable differential measurements to be performed.

Preferably, each optical fiber is connected to a connector that is connected via one or more link optical fibers to the luminescence detector system.

Advantageously, the luminescence detector system includes at least two detection channels on two different spectrum bands, one of which is the narrow band (BE) of GaN material.

According to a characteristic of the invention, the luminescence detector system includes, downstream from the optical selector device, a photodetector unit comprising one or more photomultiplier tubes.

In a variant embodiment, the optical selector device comprises a bandpass optical filter centered on the emission peak in the narrow band (BE) of GaN material.

In another embodiment, the optical selector device comprises a dispersive or diffractive optical system enabling the spectral components of the signal to be separated prior to being detected on two distinct spectrum channels using at least two photomultiplier tubes, one of which serves to detect the narrow band (BE) and is preferably provided with a narrow slit.

In another embodiment, the optical selector device is preferably constituted by a collimator lens and by a dispersive or diffractive optical system that delivers the signal to the photoelectrical converter unit made with the help of multichannel photomultiplier tubes.

In a preferred application, the luminescence detector system includes means for synchronizing the time window on the high-energy pulse shots relating to radio therapy treatment.

Various other characteristics appear from the description given below with reference to the accompanying drawings that show, as non-limiting examples, various embodiments of the invention.

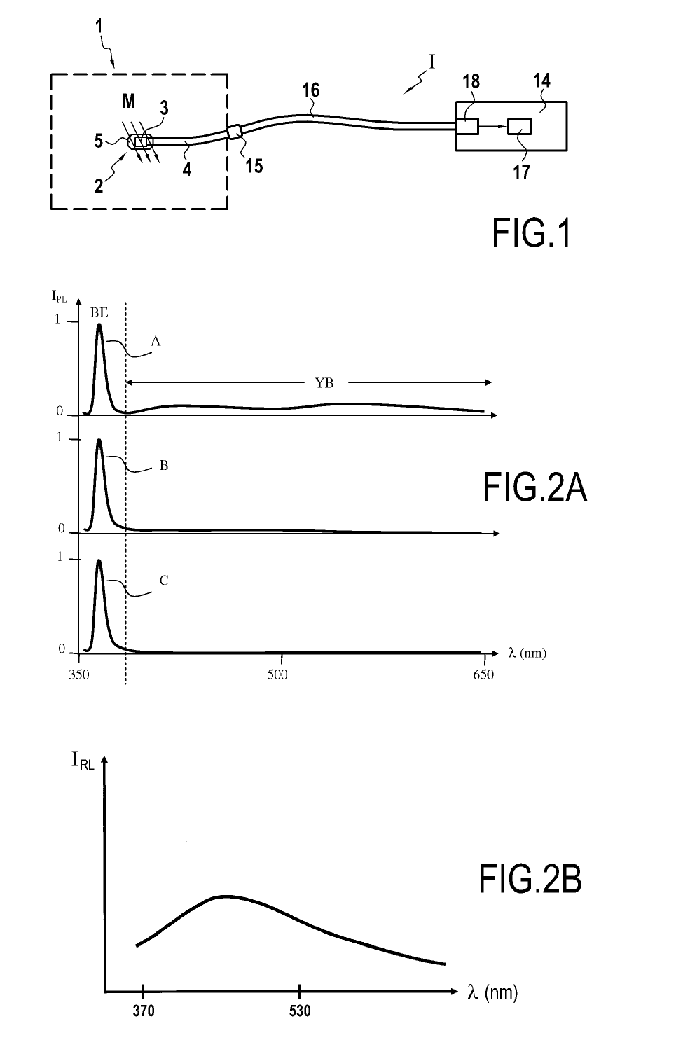

FIG. 1 is a block diagram showing a miniaturized probe in accordance with the invention.

FIG. 2A shows the typical photoluminescent emission spectrum of type n GaN for various levels of silicon doping (relative intensity I_{PL} of luminescence as a function of wavelength λ in nanometers).

FIG. 2B shows the radioluminescence spectrum of a polymer-clad fused-silica optical fiber (ETFB) (intensity I_{PL} of radioluminescence as a function of wavelength λ in nanometers).

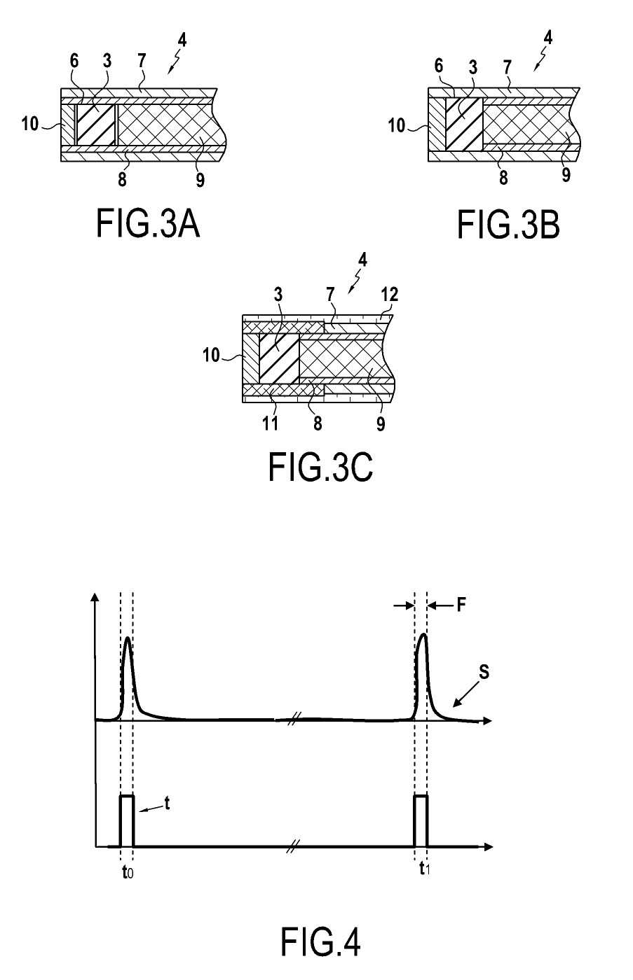

FIGS. 3A to 3C show various ways of encapsulating gallium nitride (GaN) in a miniaturized probe in accordance with the invention.

FIG. 4 shows the time response of radioluminescence to pulses of irradiation shots coming from a chemical linear accelerator.

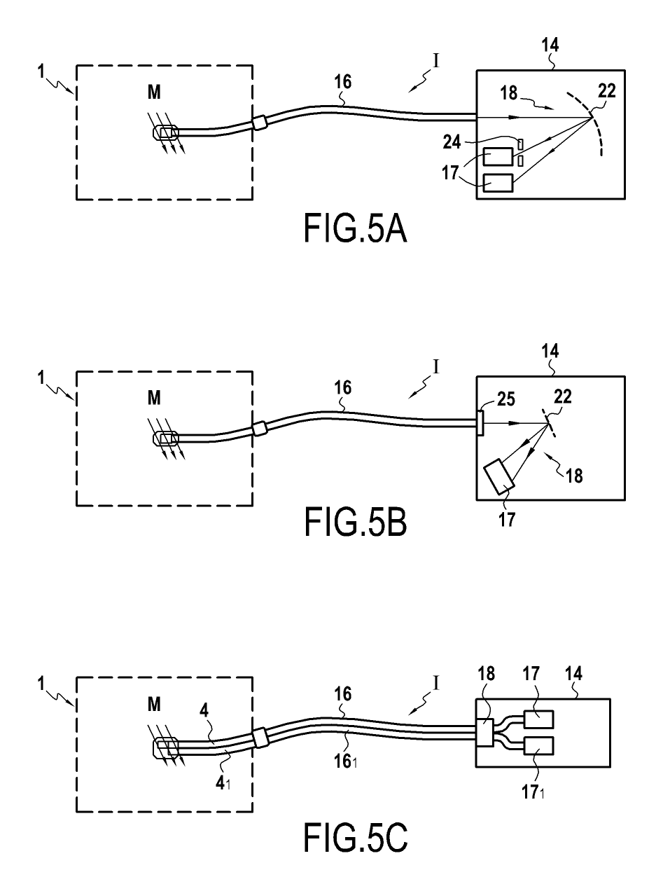

FIGS. 5A to 5C are diagrams showing various embodiments of the measurement device in accordance with the invention.

As can be seen more clearly in FIG. 1, the invention relates to a miniaturized probe 1 forming part of a dosimetry device I serving to measure high-energy radiation M such as X-rays, gamma-rays, electrons, positrons, and other high-energy particles. The probe 1 has a converter cell 2 containing radioluminescent material 3 that emits a luminescence signal of intensity that is a function of the high-energy radiation M irradiating said material. The luminescence signal is recovered by at least one optical fiber 4.

In accordance with the invention, the radioluminescent material 3 is gallium nitride GaN, a direct gap III-V semiconductor (having gap energy Eg≈3.4 electron volts (eV) at 300 K) of atomic number (Z=19) that is close to that of biological tissues. This intrinsic or non-intentionally-doped monocrystalline material typically possesses a luminescence spectrum at ambient temperature in two distinct spectrum bands, namely a narrow emission band or band edge (BE) and a broad band or yellow band (YB).

When the GaN material is subjected to high-energy radiation, electron-hole pairs are created in the material. The radiative recombination of these carriers through a plurality of channels is predominant and guarantees good radioluminescent efficiency for GaN and a response time that is very short (of nanosecond order). The emission peak in the narrow band BE that corresponds to the band gap of the material is centered around a wavelength of 365 nanometers (nm) (FIG. 2A), while the broad band (YB) emission due to defects of the material takes place at longer wavelengths and needs to be minimized in the intended applications. The emission of radioluminescence exclusively in the BE band facilitates implementing spectral discrimination of the useful signal relative to the broad band emission of the optical fiber 4 as shown in FIG. 2B. As a result, the probe 1 is advantageously made by using a GaN material that has been specially doped so that the radioluminescent emission is situated essentially in the narrow band BE of GaN material. For example, the GaN material is of the n type, being highly doped with silicon (density of dopants greater than 10¹⁹ atoms per cubic centimeter (cm³)). As can be seen in FIG. 2A, the distribution of the luminescent emission in the two emission bands of GaN (BE and YB) depends on the doping level of the material. Thus, at ambient temperature, a material that is essentially not doped or that has a low level of silicon doping possesses a luminescence spectrum of a form that is similar to curve A, whereas materials that have doping at a medium level (≈10¹⁸ atoms per cm³) and at a high level (≈10¹⁹ atoms per cm³) present spectra having approximately the appearances shown by curves B and C, respectively.

In a first variant embodiment, the GaN material 3 is encapsulated in the converter cell 2 mounted at one of the ends of the optical fiber 4 so as to constitute an invasive probe that is particularly well suited for in vivo dosimetry for radiotherapy and radiodiagnosis purposes. For example, the converter cell 2 is provided with a coating 5 adapted to fasten the GaN material 3 mechanically to the end of the optical fiber. The material chosen for the coating 5 may take account of constraints associated with the application (e.g. biocompatibility for medical dosimetry) and with optimizing the collection of the radioluminescence signal by the optical fiber. By way of example, the coating 5 is made of ETFE, polyamide, PEEK, or any other coating commonly used in invasive medical devices.

FIGS. 3A to 3C show various other ways of mounting the GaN material 3 to the end of the optical fiber 4.

In the examples shown in FIGS. 3A and 3B, the GaN material 3 is housed in a cavity 6 formed at the end of the optical fiber 4 that has a tubular protective covering 7 surrounding optical cladding 8 of the optical fiber 4. This cladding 8 is in contact with the core 9 of the optical fiber 4. In the example shown in FIG. 3A, the cavity 6 is formed directly in the core 9 of the optical fiber 4 by wet or dry etching or by any other appropriate method. Thus, the core 9 of the optical fiber 4 is removed over a determined length from the free end of the fiber, while leaving intact the cladding 8 and the protective covering 7 all the way to the free end of the optical fiber 4. Thus, an entire face of the GaN material 3 is in contact with the core 9 of the optical fiber 4. In this configuration, the cladding 8 surrounds the GaN material 3 and as a result improves the efficiency with which the luminescence signal is collected by the optical fiber 4.

In the embodiment shown in FIG. 3B, the core 9 of the fiber 4 and the cladding 8 are removed over a determined length from the free end of the fiber, while leaving the protective cover 7 intact as far the free end of the fiber. In this example, the GaN material 3 is in contact with the end of the core 9 and with the cladding 8.

In the examples described in FIGS. 3A and 3B, it should be observed that the cavity 6 possesses a length (along the axis of the optical fiber 4) that is longer than the GaN material 3 so as to enable a protective material 10 to be inserted in the end of the optical fiber 4 and close to it. This protective material 10 of biocompatible type serves to hold the GaN material 3 captive in the cavity 6 by being inserted in the end section of the cladding 8 (FIG. 3A) or of the protective covering 7 (FIG. 3B).

These variant embodiments enable the GaN material 3 to be mounted in sealed manner in line with the optical fiber without increasing the outside diameter of the optical fiber 4. For example, for in vivo dosimetry, the outside diameter of the probe may be less than 800 micrometers (μm).

FIG. 3C shows another embodiment in which the cavity 6 that receives the GaN material 3 is constituted by a tube 11 fitted over the end of the optical fiber on the cladding 8, and closed by a protective material 10. The tube 11 and the covering 7 may be inserted inside a protective jacket 12.

The interface between the GaN material 3 and the end of the optical fiber 4 may include means that are suitable for optimizing the efficiency of optical coupling, such as for example index-matching gels, and/or a graded index (GRIN) lens.

The optical fiber 4 receives the radioluminescence signal emitted by the GaN material 3 and conveys it to an electroluminescence detection system 14.

According to an embodiment characteristic, the optical fiber 4 is connected via a connector 15 to a link optical fiber 16 that is connected to the luminescence detection system 14. The probe 1 with the optical fiber 4 thus forms a single-use device that can be discarded. The link optical fiber 16 may be several tens of meters long and serves to keep the luminescence detection system 14 away from the measurement probe 1, and thus away from the irradiation zone.

The luminescence detection system 14 includes means for synchronizing the time window F on the radiation pulse shots of the radiotherapy treatment. Thus, as can be seen in FIG. 4, the luminescence signal S is taken into account from the beginning of the high-energy shot t irradiating the treatment zone in question. Typically, each high-energy shot t possesses a duration of 5 microseconds (μs). Each time window F enables the radioluminescence signal S to be taken into account at the beginning tᵢ of each shot t and throughout the duration of each shot t. This time windowing enables the signal-to-noise ratio of radioluminescence detection to be improved significantly, and thus enables measurement accuracy to be improved. For example, analyzing the radioluminescence signal over time in association with a detection system that is fast can make it possible to distinguish between the different contributions to the luminescence on the basis of their time properties, and to be unaffected by slow parasitic scintillation.

In conventional manner, the luminescence detection system 14 comprises a photodetector unit 17 performing photoelectric conversion of the luminescence signal, thereby enabling the high-energy radiation that irradiates the GaN material 3 to be measured. According to a characteristic of the invention, the photodetector unit 17 comprises one or more photomultiplier tubes having one or more paths (multianode tubes). The advantage of having recourse to a photomultiplier tube as a photodetector is obtaining high detection sensitivity and short response times so as to have good time resolution concerning the detected signal.

According to another characteristic of the invention, the luminescence detection system 14 includes an optical device 18 that serves to select the narrow emission band BE of GaN material 3. This optical device 18 is implemented by any suitable optical means and it is located upstream from the photodetector unit 17.

In an embodiment, the optical selector device 18 is constituted by a bandpass optical filter receiving the luminescence signal conveyed by the link optical fiber 16. The bandpass optical filter is centered on the emission peak in the BE band of the GaN material 3, i.e. around 365 nm. The signal as filtered in this way is detected by the photodetector unit 17 constituted by a photomultiplier tube.

This implementation is advantageously applicable when the parasitic contribution in the BE emission band of the GaN material is negligible, e.g. for highly localized irradiation (weak field). In this configuration, the volume of the optical fiber that is irradiated is small enough to ensure that the associated emission of radioluminescence is negligible compared with that from the GaN material in the passband of the filter.

FIG. 5A shows another embodiment of the system 14 for detecting the radioluminescence conveyed by the link optical fiber 16. The luminescence signal is directed towards an optical selector device 18 formed by a dispersive or diffractive optical system 22 (diffraction grating, prism, etc.) or, as shown, a concave reflection grating. The spectral components of the signal as separated spatially by such an optical system 22 are detected on two distinct spectrum channels using at least two photomultiplier tubes 17. Advantageously, in front of the photomultiplier tube 17 that is used for detecting the useful component (BE band of GaN), it is possible to place a narrow slit 24 that serves to increase spectral selectivity. This variant serves to measure both the luminescence of the GaN material in the BE band and also to estimate the contribution of parasitic luminescence in a distinct spectrum band.

FIG. 5B shows another variant embodiment of a luminescence detector system 14. The luminescence signal conveyed by the optical fiber 16 is directed to the optical selector device 18 that is preferably constituted by a collimator lens 25 and by a dispersive or diffractive optical system 22 that sends the signal to the photodetector unit 17 made using multichannel (multianode) photomultiplier tubes. This configuration enables the spectrum of the luminescence signal coming from the probe to be obtained. On the basis of this spectral information, it is possible to estimate the intensity of the luminescence of the GaN material by subtracting the parasitic contribution. This spectral analysis makes it possible to improve the estimate of the various contributions of the useful and parasitic signals in comparison with a method using two channels as shown in FIG. 5A.

It should be observed that the embodiments described above use only one optical fiber channel and that they enable the parasitic luminescence contributions to be rejected by making use of information in the spectral domain. That approach has the advantage of being well adapted to making a miniature probe.

FIG. 5C shows another embodiment that enables differential measurements to be performed. In this embodiment, the device has a reference optical fiber 4₁ and a reference link optical fiber 16₁ that are identical respectively to the optical fiber 4 and to the link optical fiber 16. The reference optical fiber 4₁ is not connected to any radioluminescent material, while the reference link optical fiber 16₁ is connected to a photomultiplier tube 17₁ that is identical to the photomultiplier tube 17. Naturally, upstream from the photomultiplier tubes 17 and 17₁, there is interposed one or other of the embodiments of the optical selector device 18.

One of the advantages of the embodiment described in FIG. 5C is that it enables the parasitic contribution to be subtracted from the useful signal in order to improve measurement accuracy and stability.

It should be assumed that the probe 1 in accordance with the invention can be miniaturized insofar as it is made essentially of an optical fiber having placed at the end thereof the detection cell that essentially comprises GaN material in accordance with the invention. This probe is inexpensive and may therefore be discardable. The probe is particularly suitable for constituting an invasive probe for single use. Another advantage of such a device lies in the possibility of measuring the high-energy radiation dose in real time. Such a device makes it possible to avoid having electrical connections between the detection cell 2 and the photomultiplier tube 17.

The invention is not limited to the examples described and shown since various modifications may be made thereto without going beyond its ambit.

What is claimed is:

1. A device for in vivo dosimetry, the device comprising:
a miniature probe (1) comprising at least:
a radioluminescent material (3) that emits a radioluminescence signal of intensity that is a function of the high-energy radiation irradiating said material; and
an optical fiber (4, 16) receiving the luminescence signal and conveying it to a luminescence detector system (14); and
a luminescence detector system (14);
the device being characterized in that the radioluminescent material (3) is gallium nitride (GaN) that emits a luminescence signal at least in a narrow band (BE), and in that the luminescence detector system (14) includes an optical device (18) enabling the narrow emission band BE of gallium nitride to be selected.

2. A device according to claim 1, characterized in that the GaN radioluminescent material is doped specifically so that it emits essentially in the narrow emission band (BE).

3. A device according to claim 1, characterized in that the GaN radioluminescent material is placed in a detection cavity (6) mounted on the end of an optical fiber or made at one of the ends of an optical fiber in order to form an invasive probe.

4. A device according to claim 3, characterized in that the optical fiber (4) includes a tubular covering (7) for protecting optical cladding (8) that contains the core (9) of the optical fiber, the core (9) of the fiber and optionally the cladding (8) being removed over an end portion of the optical fiber in order to constitute the cavity (6) for receiving the radioluminescent material (3), this cavity being closed by a protective material (10).

5. A device according to claim 1, characterized in that the probe (1) includes a reference optical fiber (41) identical to the fiber (4) connected to the luminescent material (3), but not connected to any radioluminescent material, the optical fiber (4) and the reference optical fiber (41) being connected via the optical selector device (18) to two identical photomultiplier tubes (17, 171) to enable differential measurements to be performed.

6. A device according to claim 1, characterized in that each optical fiber (4, 41) is connected to a connector (15) that is connected via one or more link optical fibers (16, 161) to the luminescence detector system (14).

7. A device according to claim 1, characterized in that the luminescence detector system (14) includes at least two detection channels on two different spectrum bands, one of which is the narrow band (BE) of GaN material.

8. A device according to claim 1, characterized in that the luminescence detector system (14) includes, downstream from the optical selector device (18), a photodetector unit (17) comprising one or more photomultiplier tubes.

9. A device according to claim 1, characterized in that the optical selector device (18) comprises a bandpass optical filter centered on the emission peak in the narrow band (BE) of GaN material (3).

10. A device according to claim 1, characterized in that the optical selector device (18) comprises a dispersive or diffractive optical system (22) enabling the spectral components of the signal to be separated prior to being detected on two distinct spectrum channels using at least two photomultiplier tubes (17), one of which serves to detect the narrow band (BE) and is preferably provided with a narrow slit (24).

11. A device according to claim 1, characterized in that the optical selector device (18) is preferably constituted by a collimator lens (25) and by a dispersive or diffractive optical system (22) that delivers the signal to the photoelectrical converter unit (17) made with the help of multichannel photomultiplier tubes.

12. A device according to claim 1, characterized in that the luminescence detector system (14) includes means for synchronizing the time window (F) on the high-energy pulse shots (t) relating to radio therapy treatment.
